(19) United States
(12) Patent Application Publication
(10) Pub. No.: US 2018/0344636 A1
(43) Pub. Date: Dec. 6, 2018

(54) METHOD FOR MANUFACTURING AN ANTI-VAGINITIS SUPPOSITORY

(71) Applicant: Feng Chia University, Taichung City (TW)

(72) Inventor: Tse-Hao KO, Taichung (TW)

(73) Assignee: Feng Chia University, Taichung City (TW)

(21) Appl. No.: 15/615,271

(22) Filed: Jun. 6, 2017

(51) Int. Cl. A61K 9/02; A61K 33/38

(52) U.S. Cl. CPC A61K 9/02; A61K 33/38

(57) ABSTRACT

A method for manufacturing an anti-vaginitis suppository includes: immersing a carbonaceous material in an aqueous solution containing an active ingredient salt; thermally drying the aqueous solution at 60-500° C. to attach an agglomerate of the active ingredient salt to the carbonaceous material; pyrolyzing the agglomerate at 200-1000° C. to convert into a particle of the active ingredient attached to the carbonaceous material; mixing the carbonaceous material and the particle thereon with a matrix to obtain a mixture; and solidifying the mixture to form the suppository.

### FIELD OF THE INVENTION

The present invention is directed to a method for manufacturing a suppository, and more particularly to a method for manufacturing an anti-vaginitis suppository.

### BACKGROUND OF THE INVENTION

Vaginitis is a women's common disease, and according to the contributing causes of the disease, it includes: bacterial vaginitis, mycotic vaginitis, trichomonas vaginitis, senile vaginitis, and infantile vaginitis. If this disease is not properly treated, some symptoms, e.g. vaginal wounds or uterine cervical erosion, may occur. The medicine currently used for treatment of vaginitis includes an external-antibiotic medicine and an internal-antibiotic medicine. The external-antibiotic medicine is mostly in the form of a lotion, a suppository, or an effervescent tablet. These dosage forms can confer therapeutic effects, but the antibiotic is prone to develop resistance and side effects.

China Patent Publication No. CN105434334A discloses a method for manufacturing a non-antibiotic suppository. Specifically, it is a method for manufacturing a suppository with an activated carbon, and the detailed steps are described as follows. Firstly, after mixed well and heated to 65-75° C., 98-102 parts of water, 0.4-0.6 parts of nano-silver aqueous solution, and 0.8-1.2 parts of carbomer are mixed well with 5-7 parts of fatty acid glyceride to obtain a mixture. Secondly, after 3-5 parts of carboxymethyl cellulose, 6-9 parts of hydroxypropyl cellulose, and 78-82 parts of activated carbon are mixed well and then added to the foregoing mixture, the mixture is stirred into dough. Thirdly, the dough is pressed into a mold, cooled, and released from the mold. Fourthly, the dough is heated at 65-85° C. for 2-6 hours, and then heated at 105-115° C. for 0.5-2.5 hours. Finally, the dough is packaged with absorbent gauze. By the prior method, some nano-silver is attached to the activated carbon in the manufactured suppository, and some is dissociated therein. This causes such a low bactericidal or bacteriostatic activity of the suppository that it is inefficiently used for treatment of vaginitis. Furthermore, the dissociated nano-silver can elicit allergic response and toxicity. Additionally, the foregoing manufacturing method cannot make the suppository quality maintained at a certain level, so it is unsuitable for a large scale production.

Therefore, an improvement on the prior manufacturing method is desirable.

### SUMMARY OF THE INVENTION

An objective of the present invention is to provide a method for manufacturing an anti-vaginitis suppository, and the provided method includes: immersing a carbonaceous material in an aqueous solution containing an active ingredient salt; thermally drying the aqueous solution at 60-500° C. to attach an agglomerate of the active ingredient salt to the carbonaceous material; pyrolyzing the agglomerate at 200-1000° C. to convert into a particle of the active ingredient attached to the carbonaceous material; mixing the carbonaceous material and the particle thereon with a matrix to obtain a mixture; and solidifying the mixture to form the suppository.

### BRIEF DESCRIPTION OF THE DRAWINGS

FIG. 1 is a flowchart illustrating a method for manufacturing a suppository in an embodiment.

### DETAILED DESCRIPTION OF THE INVENTION

The detailed description and preferred embodiments of the invention will be set forth in the following content, and provided for people skilled in the art so as to understand the characteristics of the invention.

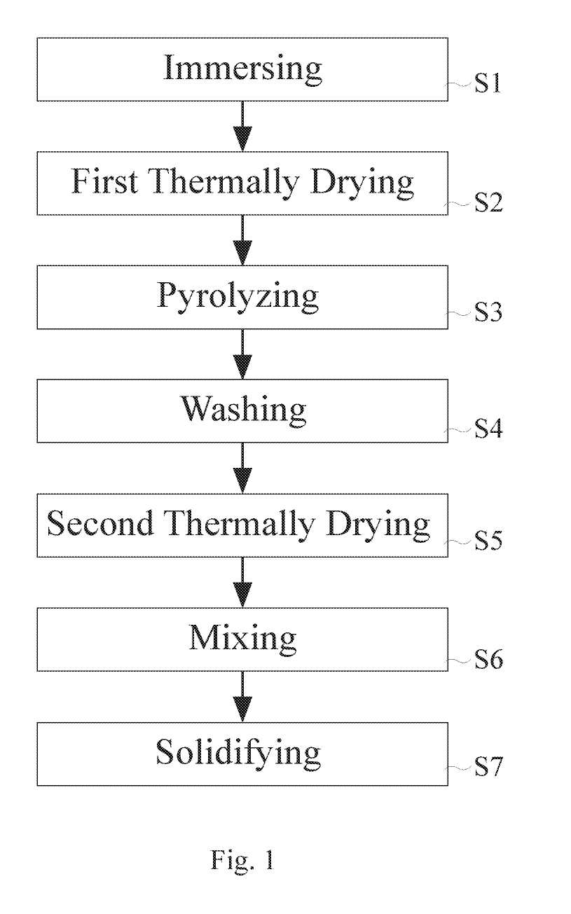

An embodiment of the present invention provides a method for manufacturing a suppository, and the suppository has high bactericidal or bacteriostatic activity so as to be used in treatment of vaginitis. As shown in FIG. 1, the method comprises the following steps: immersing (S1), first thermally drying (S2), pyrolyzing (S3), washing (S4), second thermally drying (S5), mixing (S6), and solidifying (S7).

First, the immersing step (S1) is immersing a carbonaceous material in an aqueous solution containing an active ingredient salt, which can make the carbonaceous material contact the salt. This step (S1) may be implemented under a room temperature and pressure, a vacuum, or a stir. The vacuum condition and the stir condition can prevent capillary action so that the salt is uniformly distributed in the aqueous solution. When this step (S1) is practiced, the carbonaceous material may be soaked in the aqueous solution for more than 1 minute, preferably for 1-800 minutes, and more preferably for 5-240 minutes. In order to allow the salt to be uniformly distributed in the aqueous solution, the aqueous solution may further has a polar solvent, such as an alcohol, an aldehyde, a ketone, or an ether; based on the total weight of the water and the polar solvent, the polar solvent may be present in an amount of 5 wt %-70 wt %. Furthermore, an example of the carbonaceous material is but not limited to an activated carbon fiber, a carbon fiber, an activated carbon powder, a charcoal, a bamboo charcoal granule, a carbon black, a graphite powder, a carbon nanotube, a carbon nanopowder, a graphene, a swelling graphite powder, a carbon powder made from phenol formaldehyde resins, or a carbon powder made from artificial resins. An example of the salt is but not limited to a silver salt, a copper salt, a gold salt, a palladium salt, a zinc salt, a platinum salt, an aluminum salt, a nickel salt, a cobalt salt, a silicon salt, a calcium salt, a titanium salt, or a chromium salt, and the concentration thereof is preferably of 0.00001-20 M. For example, the salt is an acetate of the active ingredient (e.g. silver acetate), a nitrate of the active ingredient (e.g. silver nitrate, copper nitrate, or zinc nitrate), a phosphate of the active ingredient (e.g. silver phosphate), or a sulfonate of the active ingredient (e.g. silver sulfonate).

Next, the first thermally drying step (S2) is thermally drying the aqueous solution at 60-500° C., which can attach an agglomerate of the active ingredient salt to the carbonaceous material. When this step (S2) is practiced, the aqueous solution may be stayed at 60-500° C. for more than 1 minute, preferably for 1-800 minutes, and more preferably for 5-240 minutes.

After, the pyrolyzing step (S3) is pyrolyzing the agglomerate at 200-1000° C., which can convert the agglomerate into a particle of the active ingredient attached to the carbonaceous material. Based on the weight of the carbonaceous material, the particle may be present in an amount of less than 50 wt %, and preferably of 0.0001 wt %-25 wt %. This step (S3) may be executed under a vacuum, a nitrogen gas, or an inert gas. Additionally, when this step (S3) is practiced, the agglomerate may be stayed at 200-1000° C. for more than 1 minute, preferably for 1-800 minutes, and more preferably for 5-240 minutes.

Then, the washing step (S4) is washing the carbonaceous material with water, which can remove any dissociated particle so that the subsequently obtained suppository has a low dissociated particle amount. When this step (S4) is performed, the carbonaceous material may be soaked in the water or rinsed with the water for more than 1 minute, preferably for 5-480 minutes, and more preferably for 10-240 minutes.

Subsequently, the second thermally drying step (S5) is thermally drying the carbonaceous material at 60-500° C., which can remove any remaining liquid. When this step (S5) is practiced, the carbonaceous material may be stayed at 60-500° C. for more than 1 minute, preferably for 1-800 minutes, and more preferably for 5-240 minutes.

Afterward, the mixing step (S6) is mixing the carbonaceous material and the particle thereon with a matrix to obtain a mixture. Based on the weight of the matrix, the carbonaceous material and the particle are totally present in an amount of 0.001 wt %-20 wt %. An example of the matrix is but not limited to an oil matrix or a water-soluble and hydrophilic matrix. For example, the oil matrix is a cocoa butter, a semi-synthetic fatty acid glyceride, or a fully synthetic fatty acid glyceride; the semi-synthetic fatty acid glyceride is such as a semi-synthetic coconut oil, a semi-synthetic litsea cubeba oil, or a semi-synthetic palm oil, and the fully synthetic fatty acid glyceride is such as a propanediol distearate. For example, the water-soluble and hydrophilic matrix is a glycerogelatin or a polyethylene glycol. Moreover, in order to enhance bactericidal or bacteriostatic activity of the subsequently obtained suppository, when this step (S6) is performed, an antibiotic may be further mixed with the carbonaceous material, the particle, and the matrix, such as metronidazole, clindamycin, butoconazole, clotrimazole, miconazole, nystatin, tioconazole, terconazole, or econazole.

Finally, the solidifying step (S7) is solidifying the mixture to form the suppository. When this step (S7) is executed, the mixture may be stayed at a low temperature.

The following examples are offered to further illustrate the present invention:

### Example 1

First of all, a fabric of polyacrylonitrile (PAN)-based activated carbon fibers (Taiwan Carbon Technology Co., Ltd) was prepared, the characteristics thereof included: a BET surface area of 1,600 m²/g, a density of 2.09 g/cm³, and a carbon content of 85 wt %. The fiber fabric was soaked in 0.1 M silver nitrate aqueous solution for 5 hours, and then thermally dried at 80° C. for 2 hours. By this way, an agglomerate was formed with silver nitrate and attached to the fiber fabric. Afterwards, the agglomerate was pyrolyzed at 400° C. in the presence of nitrogen gas to convert into a silver particle on the fiber fabric. Next, the fiber fabric was sequentially washed, dried, ground, and sieved to obtain a composition. The composition had an activated carbon powder and the silver particle attached to the powder, and the characteristics thereof included: a BET surface area of 1,220 m²/g, a density of 2.13 g/cm³, and a silver content of 0.03 wt %. After 10 g of fatty acid glyceride, 5 g of hydroxypropyl cellulose, and 1 g of the composition were mixed, the obtained mixture was heated to 65° C. and pressed into a mold. Finally, the mixture was cooled to obtain suppositories. Each one weighed 2.5 g and contained 0.156 g of the composition.

### Example 2

First of all, a fabric of polyacrylonitrile-based activated carbon fibers (Taiwan Carbon Technology Co., Ltd) was prepared, the characteristics thereof included: a BET surface area of 1,600 m²/g, a density of 2.09 g/cm³, and a carbon content of 85 wt %. The fiber fabric was soaked in 0.1 M silver nitrate aqueous solution and 0.1 M copper nitrate aqueous solution for 5 hours, and then thermally dried at 80° C. for 2 hours. By this way, an agglomerate was formed with silver nitrate and copper nitrate, and the agglomerate was attached to the fiber fabric. Afterwards, the agglomerate was pyrolyzed at 400° C. in the presence of nitrogen gas to convert into a silver particle and a copper particle on the fiber fabric. Next, the fiber fabric was sequentially washed, dried, ground, and sieved to obtain a composition. The composition had an activated carbon powder, and the silver particle and the copper particle both attached to the powder, and the characteristics thereof included: a BET surface area of 1,200 m²/g, a density of 2.13 g/cm³, a silver content of 0.03 wt %, and a copper content of 0.05 wt %. After 10 g of fatty acid glyceride, 5 g of hydroxypropyl cellulose, and 1 g of the composition were mixed, the obtained mixture was heated to 65° C. and pressed into a mold. Finally, the mixture was cooled to obtain suppositories. Each one weighed 2.5 g and contained 0.156 g of the composition.

### Example 3

First of all, a plant-based activated carbon powder was prepared; a BET surface area thereof was of 800 m²/g. The activated carbon powder was soaked in 0.1 M silver nitrate aqueous solution under a vacuum for 5 hours, and then thermally dried at 80° C. for 2 hours. By this way, an agglomerate was formed with silver nitrate and attached to the activated carbon powder. Afterwards, the agglomerate was pyrolyzed at 400° C. in the presence of nitrogen gas to convert into a silver particle on the activated carbon powder. Next, the activated carbon powder was sequentially washed, dried, ground, and sieved to obtain a composition. The composition had the activated carbon powder and the silver particle attached to the powder, and the characteristics thereof included: a BET surface area of 780 m²/g and a silver content of 0.02 wt %. According to the weight of the subsequently obtained mixture, 17% of gelatin and 28% of water were mixed well, and then they were mixed with 54% of glycerol and 1% of the composition. After which, the obtained mixture was heated to 65° C. and pressed into a mold. Finally, the mixture was cooled to obtain suppositories. Each one weighed 15 g and contained 0.15 g of the composition.

### Comparative Example 1

10 g of fatty acid glyceride, 5 g of hydroxypropyl cellulose, and 1 g of polyacrylonitrile-based activated carbon fibers were mixed. After which, the obtained mixture was heated to 65° C. and pressed into a mold. Finally, the mixture was cooled to obtain suppositories. Each one weighed 2.5 g and contained 0.156 g of the activated carbon fibers.

### Comparative Example 2

According to the weight of the subsequently obtained mixture, 17% of gelatin and 28% of water were mixed well, and then they were mixed with 54% of glycerol and 1% of plant-based activated carbon powders. After which, the obtained mixture was heated to 65° C. and pressed into a mold. Finally, the mixture was cooled to obtain suppositories. Each one weighed 15 g and contained 0.15 g of the powders.

### Comparative Example 3

This comparative example was implemented with reference to China Patent Publication No. CN105434334A. First, 0.5 g of nano-silver aqueous solution was mixed well with 100 g of water. After being heated to 70° C., 6 g of fatty acid glyceride was added into the aqueous solution to give a mixture. After which, 4 g of carboxymethyl cellulose, 7 g of low-substituted hydroxypropyl cellulose, and 80 g of activated carbon powders were mixed well with the mixture to form dough. Finally, the dough was pressed into a mold and cooled to obtain suppositories.

### Analysis

According to the American Association of Textile Chemists and Colorists (AATCC)-100 antibacterial product test, various bacterial strains were cultured with 0.2 g of each of all the suppositories for 24 hours to perform an antibacterial analysis. As the results shown in Table 1, each suppository in Examples 1-3 has better antibacterial activity than each suppository in Comparative Examples 1-3. From the results of Examples 1-3 and Comparative Examples 1-2, it is found that silver particles or copper particles can be converted into cations (Ag⁺ or Cu²⁺/Cu⁺) and these ions can eliminate bacteria. From the results of Examples 1-3 and Comparative Example 3, it is found that the different manufacturing processes can confer distinct bactericidal or bacteriostatic activity for these suppositories containing silver.

TABLE 1
Antibacterial activity of each suppository
after 24-hour bacterialculture

Sterilizing Rate(%)

| | Candida | Escherichia | Staphylococcus |
|---|---|---|---|
| | albicans | coli | aureus |
| | (ATCC18814) | (ATCC8739) | (ATCC6538P) |
| Example 1 | 95.82 | 94.32 | 93.75 |
| Example 2 | 98.75 | 98.63 | 97.52 |
| Example 3 | 93.37 | 93.25 | 92.52 |
| Comparative | 70.22 | 67.42 | 52.34 |
| Example 1 | | | |
| Comparative | 52.67 | 40.35 | 35.37 |
| Example 2 | | | |
| Comparative | 73.22 | 74.83 | 62.54 |
| Example 3 | | | |

As described above, the manufacturing method of the foregoing embodiment can lead to uniform distribution of the active ingredient particle on the carbonaceous material. Therefore, when the manufactured suppository is placed in a vagina, a uniform contact of the particle with the vagina exists. In another aspect, this method can reduce the amount of the dissociated particle so that the allergic response or toxicity elicited thereby cannot occur when the suppository is placed in a vagina. In still another aspect, the described method can make the suppository quality maintained at a certain level so as to be suitable for a large scale production.

While the invention has been described in connection with what is considered the most practical and preferred embodiments, it is understood that this invention is not limited to the disclosed embodiments but is intended to cover various arrangements included within the spirit and scope of the broadest interpretation so as to encompass all such modifications and equivalent arrangements.

What is claimed is:

1. A method for manufacturing an anti-vaginitis suppository, comprising:
immersing a carbonaceous material in an aqueous solution containing an active ingredient salt;
thermally drying the aqueous solution at 60-500° C. to attach an agglomerate of the active ingredient salt to the carbonaceous material;
pyrolyzing the agglomerate at 200-1000° C. to convert into a particle of the active ingredient attached to the carbonaceous material;
mixing the carbonaceous material and the particle thereon with a matrix to obtain a mixture; and
solidifying the mixture to form the suppository.

2. The method as claimed in claim 1, between the pyrolyzing step and the mixing step, further comprising:
washing the carbonaceous material with water; and
thermally drying the carbonaceous material at 60-500° C.

3. The method as claimed in claim 1, wherein the immersing step is implemented under a room temperature and pressure, a vacuum, or a stir.

4. The method as claimed in claim 1, wherein the carbonaceous material is selected from the group consisting of an activated carbon fiber, a carbon fiber, an activated carbon powder, a charcoal, a bamboo charcoal granule, a carbon black, a graphite powder, a carbon nanotube, a carbon nanopowder, a graphene, a swelling graphite powder, a carbon powder made from phenol formaldehyde resins, and a carbon powder made from artificial resins.

5. The method as claimed in claim 1, wherein the salt is selected from the group consisting of a silver salt, a copper salt, a gold salt, a palladium salt, a zinc salt, a platinum salt, an aluminum salt, a nickel salt, a cobalt salt, a silicon salt, a calcium salt, a titanium salt, and a chromium salt.

6. The method as claimed in claim 1, wherein the aqueous solution further has a polar solvent.

7. The method as claimed in claim 6, wherein the polar solvent is an alcohol, an aldehyde, a ketone, or an ether.

8. The method as claimed in claim 1, wherein the pyrolyzing step is implemented under a vacuum, a nitrogen gas, or an inert gas.

9. The method as claimed in claim 1, wherein the carbonaceous material and the particle are totally present in an amount of 0.001 wt %-20 wt % based on a weight of the matrix.

10. The method as claimed in claim 1, wherein the matrix is an oil matrix or a water-soluble and hydrophilic matrix.

11. The method as claimed in claim 10, wherein the oil matrix is a cocoa butter, a semi-synthetic fatty acid glyceride, or a fully synthetic fatty acid glyceride.

12. The method as claimed in claim 10, wherein the water-soluble and hydrophilic matrix is a glycerogelatin or a polyethylene glycol.

13. The method as claimed in claim 1, wherein the mixing step further comprises:
mixing an antibiotic with the carbonaceous material, the particle, and the matrix.

14. The method as claimed in claim 13, wherein the antibiotic is selected from the group consisting of metronidazole, clindamycin, butoconazole, clotrimazole, miconazole, nystatin, tioconazole, terconazole, and econazole.
